(19) United States
(12) Patent Application Publication
(10) Pub. No.: US 2008/0081986 A1
(43) Pub. Date: Apr. 3, 2008

(54) METHOD AND APPARATUS FOR GENERATING A MAGNETIC RESONANCE IMAGE

(75) Inventor: Glenn S. Slavin, Silver Spring, MD (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/536,917

(22) Filed: Sep. 29, 2006

(51) Int. Cl. A61B 5/05

(52) U.S. Cl. 600/410

(57) ABSTRACT

A method for generating a magnetic resonance image includes a series of inversion and saturation pulses, in which the pulses null the MRI signal from both fat and a second tissue for a single MRI acquisition. An inversion pulse is used to provide a null point for the second tissue. A pair of fat-selective saturation and inversion pulses are used to null the MRI signal from fat at approximately the same time as the second tissue reaches its null point. The saturation pulse is used to create a known state for the fat magnetization, allowing the flip angle of the fat-selective inversion pulse to be determined such that the null point of fat approximately coincides with the null point of the second tissue.

### TECHNICAL FIELD

The present invention relates generally to magnetic resonance imaging (MRI) systems and in particular, to a method and apparatus for generating a magnetic resonance image that includes suppressing the MRI signals from fat and a second tissue in the same image acquisition.

### BACKGROUND

Magnetic resonance imaging (MRI) is a medical imaging modality that can create pictures of the inside of a human body without using x-rays or other ionizing radiation. MRI uses a powerful magnet to create a strong, uniform, static magnetic field (i.e., the “main magnetic field”). When a human body, or part of a human body, is placed in the main magnetic field, the nuclear spins that are associated with the hydrogen nuclei in tissue water become polarized. This means that the magnetic moments that are associated with these spins become preferentially aligned along the direction of the main magnetic field, resulting in a small net tissue magnetization along that axis (the “z axis”, by convention). An MRI system also comprises components called gradient coils that produce smaller amplitude, spatially varying magnetic fields when current is applied to them. Typically, gradient coils are designed to produce a magnetic field component that is aligned along the z axis, and that varies linearly in amplitude with position along one of the x, y or z axes. The effect of a gradient coil is to create a small ramp on the magnetic field strength, and concomitantly on the resonant frequency of the nuclear spins, along a single axis. Three gradient coils with orthogonal axes are used to “spatially encode” the MR signal by creating a signature resonance frequency at each location in the body. Radio frequency (RF) coils are used to create pulses of RF energy at or near the resonance frequency of the hydrogen nuclei. These coils are used to add energy to the nuclear spin system in a controlled fashion. As the nuclear spins then relax back to their rest energy state, they give up energy in the form of an RF signal. This signal is detected by the MRI system and is transformed into an image using a computer and known reconstruction algorithms.

MR images may be created by applying currents to the gradient and RF coils according to known algorithms called “pulse sequences”. A pulse sequence diagram may be used to show the amplitude, phase and timing of the various current pulses applied to the gradient and RF coils for a given pulse sequence. The selection of a pulse sequence determines the relative appearance of different tissue types in the resultant images, emphasizing or suppressing tissue types as desired. The inherent MR properties of tissue, most commonly the relaxation times T1 and T2, may be exploited to create images with a desirable contrast between different tissues. For example, in an MR image of a brain, gray matter may be caused to appear lighter or darker than white matter, according to the MRI system operator's choice of pulse sequence.

A pulse sequence may include a “spin preparation”, which is comprised of RF and gradient pulses that are played out (i.e., performed or applied) prior to the acquisition of MR data. A spin preparation may be used to control the appearance of a specific tissue type in an image, or to suppress signal from a certain tissue. Tissue suppression techniques are most commonly used for suppressing signal from fat. Multiple spin preparations are known that are able to suppress signal from fat, including CHESS (Chemical Shift Selective) pulses and Inversion Recovery preparations.

In certain clinical imaging applications, it is desirable to suppress the signal not only from fat tissue but also from a second tissue in the same set of images. In cardiac MRI, for example, a paramagnetic contrast agent is used to visualize ischemically injured myocardial tissue. After a bolus of contrast agent is delivered intravenously, infarcted tissue retains a higher concentration of contrast agent for a longer period. This contrast agent shortens the T1 in the infarcted tissue, causing it to appear bright relative to healthy myocardium on T1-weighted images. Imaging the heart after a delay post injection of a contrast agent is called “myocardial delayed enhancement imaging”. Tissues that have a delayed hyper-enhancement are considered non-viable. In this type of imaging, it is desirable to choose a pulse sequence that can suppress the signal from healthy myocardium, so that the borders of the bright contrast-media-enhancing infarcted tissue may be clearly depicted. However, the presence of adjacent pericardial fat, which is also bright on a T1-weighted sequence, can negatively impact the identification of the infarct's borders.

Accordingly, it would be desirable to provide a method and apparatus for suppressing signals generated by fat tissue and a second tissue in an imaging application. In particular, it would be advantageous to provide a method and apparatus for generating a magnetic resonance image that is configured to suppress signals generated by fat tissue and a second tissue.

### BRIEF DESCRIPTION OF THE INVENTION

In accordance with an embodiment, a method for generating a magnetic resonance image includes applying a magnetic field to a subject, the subject comprising a plurality of tissues including fat tissue and a second tissue, wherein the magnetic field causes a net longitudinal magnetization in the plurality of tissues including the fat tissue and the second tissue, generating an inversion radio frequency pulse configured to invert the longitudinal magnetization from the plurality of tissues including the fat tissue and the second tissue, generating a fat-selective saturation radio frequency pulse configured to saturate the longitudinal magnetization of the fat tissue, generating a fat-selective inversion radio frequency pulse configured to invert the longitudinal magnetization of the fat tissue, generating a first excitation radio frequency pulse, and acquiring magnetic resonance imaging data.

In accordance with another embodiment, a computer-readable medium having computer-executable instructions for performing a method for generating a magnetic resonance image includes program code for generating an inversion radio frequency pulse configured to invert a longitudinal magnetization from a plurality of tissues in a subject including a fat tissue and a second tissue, program code for generating a fat-selective saturation radio frequency pulse configured to saturate a longitudinal magnetization of the fat tissue, program code for generating a fat-selective inversion radio frequency pulse configured to invert the longitudinal magnetization of the fat tissue, program code for generating a first excitation radio frequency pulse, and program code for acquiring magnetic resonance imaging data.

In accordance with another embodiment, an apparatus for generating a magnetic resonance image includes a magnetic resonance imaging assembly comprising a magnet, a plurality of gradient coils, a radio frequency coil, a radio frequency transceiver system, and a pulse generator module, and a computer system coupled to the magnetic resonance imaging assembly and programmed to perform a pulse sequence comprised of an inversion radio frequency pulse configured to invert a longitudinal magnetization from a plurality of tissues in a subject including a fat tissue and a second tissue, a fat-selective saturation radio frequency pulse configured to saturate a longitudinal magnetization of the fat tissue, a fat-selective inversion radio frequency pulse configured to invert the longitudinal magnetization of the fat tissue, a first excitation radio frequency pulse, and an acquisition window to acquire magnetic resonance imaging data.

### BRIEF DESCRIPTION OF THE DRAWINGS

Embodiments are illustrated by way of example and not limitation in the figures of the accompanying drawings, in which like reference numerals indicate corresponding, analogous or similar elements, and in which:

FIG. 1 is a schematic block diagram of an exemplary magnetic resonance imaging system;

FIG. 2 is a schematic diagram of an exemplary pulse sequence that includes a spin preparation comprising a fat-selective saturation RF pulse and a fat-selective inversion RF pulse in accordance with an embodiment;

FIG. 3 is a graph of the resulting longitudinal magnetization M_{z} for fat and for the second tissue in accordance with the spin preparation illustrated in FIG. 2 in accordance with an embodiment; and

FIG. 4 shows a pulse sequence diagram of an exemplary 2D fast gradient echo pulse sequence that uses the spin preparation illustrated in FIG. 2 to suppress MR signals from fat and healthy myocardial tissue in accordance with an exemplary embodiment.

### DETAILED DESCRIPTION

In the following detailed description, numerous specific details are set forth in order to provide a thorough understanding of embodiments. However it will be understood by those of ordinary skill in the art that the embodiments may be practiced without these specific details. In other instances, well-known methods, procedures, components and circuits have not been described in detail so as not to obscure the embodiments.

To suppress signals from fat tissue in a subject, a spin preparation may include a combination of a fat-selective saturation RF pulse and a fat-selective inversion RF pulse. Preferably, the fat-selective saturation and inversion RF pulses are inserted in the spin preparation between an inversion RF pulse (used to suppress at least a second tissue in the subject) and an acquisition window of the pulse sequence. Such a combination of fat-selective saturation and inversion RF pulses suppresses the fat signal without disturbing the desired Ti contrast that develops between the other (non-fatty) tissues of interest. The resultant spin preparation is comprised of: an inversion RF pulse configured to invert the longitudinal magnetization from all tissues including the fat tissue and the second tissue, followed by a fat-selective saturation pulse (e.g. a 90° frequency-selective RF pulse), then a delay, followed by a fat-selective inversion RF pulse with flip angle tuned for the acquisition scheme such that fat is also nulled when the magnetization from the second tissue is nulled. In this application, “nulled” is used to mean that the longitudinal magnetization of a tissue is significantly reduced, such that it no longer detracts from a reader's ability to visualize the surrounding tissue. This does not require that data is acquired at exactly the null point of the tissue, but holds for a window of time around the null point. The purpose of the saturation RF pulse is to “set” the fat magnetization to a known value so that the evolution of the fat magnetization throughout the rest of the sequence may be reliably predicted, and an appropriate flip angle for the fat-selective inversion RF pulse may be determined. Without this pulse, it is usually not possible to accurately predict the longitudinal magnetization from fat during the pulse sequence. The second tissue need only have a water resonance with a longer T1 than fat for this spin preparation to be effective.

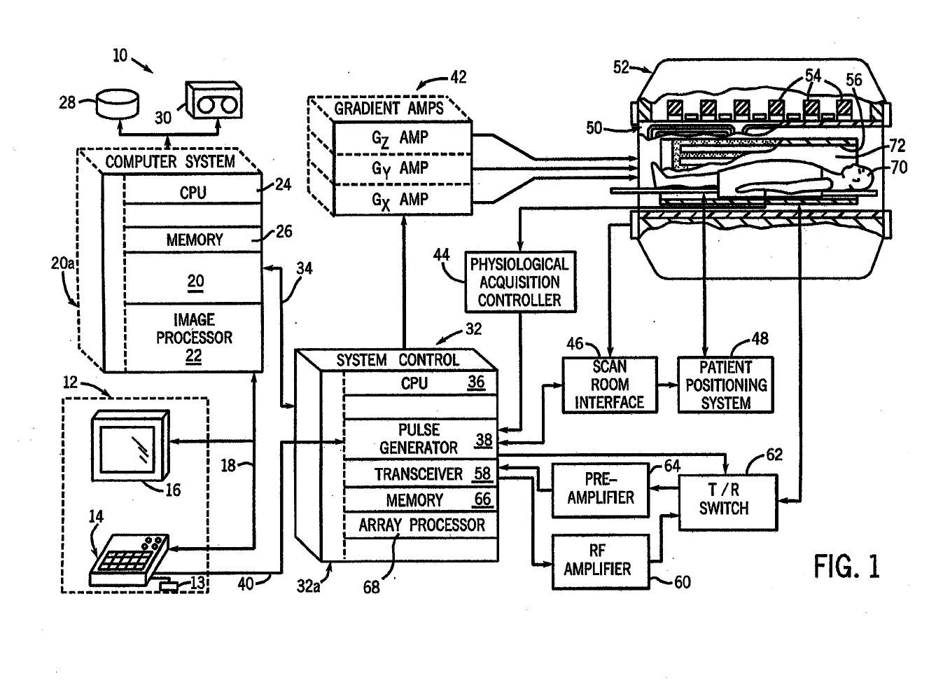

FIG. 1 is a schematic block diagram of an exemplary magnetic resonance imaging system. The operation of MRI system 10 is controlled from an operator console 12 that includes a keyboard or other input device 13, a control panel 14, and a display 16. The console 12 communicates through a link 18 with a computer system 20 and provides an interface for an operator to prescribe MRI scans, display the resultant images, perform image processing on the images, and archive data and images. The computer system 20 includes a number of modules that communicate with each other through electrical and/or data connections, for example such as are provided by using a backplane 20a. Data connections may be direct wired links, or may be fiberoptic connections or wireless communication links or the like. These modules include an image processor module 22, a CPU module 24 and a memory module 26. Memory module 26 may be, for example, a frame buffer for storing image data arrays as known in the art. In an alternative embodiment, the image processor module 22 may be replaced by image processing functionality on the CPU module 24. The computer system 20 is linked to archival media devices, such as disk storage 28 and tape drive 30 for storage of image data and programs, and communicates with a separate system control computer 32 through a high speed serial link 34. Archival media include but are not limited to: random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which may be used to store the desired instructions and which can be accessed by computer system 20, including by internet or other computer network forms of access. The input device 13 can include a mouse, joystick, keyboard, track ball, touch activated screen, light wand, voice control, or any similar or equivalent input device, and may be used for interactive geometry prescription.

The system control computer 32 includes a set of modules in communication with each other via electrical and/or data connections 32a. Data connections 32a may be direct wired links, or may be fiberoptic connections or wireless communication links or the like. In alternative embodiments, the modules of computer system 20 and system control computer 32 may be implemented on the same computer systems or a plurality of computer systems. The modules of system control computer 32 include a CPU module 36 and a pulse generator module 38 that connects to the operator console 12 through a communications link 40. It is through link 40 that the system control computer 32 receives commands from the operator to indicate the scan sequence that is to be performed. The pulse generator module 38 operates the system components that play out (i.e., perform) the desired pulse sequence and produces data called RF waveforms which control the timing, strength and shape of the RF pulses to be used, and the timing and length of the data acquisition window. The pulse generator module 38 connects to a gradient amplifier system 42 and produces data called gradient waveforms which control the timing and shape of the gradient pulses that are to be used during the scan. The pulse generator module 38 may also receive patient data from a physiological acquisition controller 44 that receives signals from a number of different sensors connected to the patient, such as ECG signals from electrodes attached to the patient. The pulse generator module 38 connects to a scan room interface circuit 46 that receives signals from various sensors associated with the condition of the patient and the magnet system. It is also through the scan room interface circuit 46 that a patient positioning system 48 receives commands to move the patient table to the desired position for the scan.

The gradient waveforms produced by the pulse generator module 38 are applied to gradient amplifier system 42 which is comprised of Gx, Gy and Gz amplifiers. Each gradient amplifier excites a corresponding physical gradient coil in a gradient coil assembly generally designated 50 to produce the magnetic field gradient pulses used for spatially encoding acquired signals. The gradient coil assembly 50 forms part of a magnet assembly 52 that includes a polarizing magnet 54 and a whole-body RF coil 56. A patient or imaging subject 70 may be positioned within a cylindrical imaging volume 72 of the magnet assembly 52. A transceiver module 58 in the system control computer 32 produces pulses that are amplified by an RF amplifier 60 and coupled to the RF coils 56 by a transmit/receive switch 62. The resulting signals emitted by the excited nuclei in the patient may be sensed by the same RF coil 56 and coupled through the transmit/receive switch 62 to a preamplifier 64. The amplified MR signals are demodulated, filtered and digitized in the receiver section of the transceiver 58. The transmit/receive switch 62 is controlled by a signal from the pulse generator module 38 to electrically connect the RF amplifier 60 to the RF coil 56 during the transmit mode and to connect the preamplifier 64 to the coil during the receive mode. The transmit/receive switch 62 can also enable a separate RF coil (for example, a surface coil) to be used in either the transmit or receive mode.

The MR signals sensed by the RF coil 56 are digitized by the transceiver module 58 and transferred to a memory module 66 in the system control computer 32. Typically, frames of data corresponding to MR signals are stored temporarily in the memory module 66 until they are subsequently transformed to create images. Most commonly, a Fourier transform is used to create images from the MR data. These images are communicated through the high speed link 34 to the computer system 20 where it is stored in memory, such as disk storage 28. In response to commands received from the operator console 12, this image data may be archived in long term storage, such as on the tape drive 30, or it may be further processed by the image processor 22 and conveyed to the operator console 12 and presented on display 16.

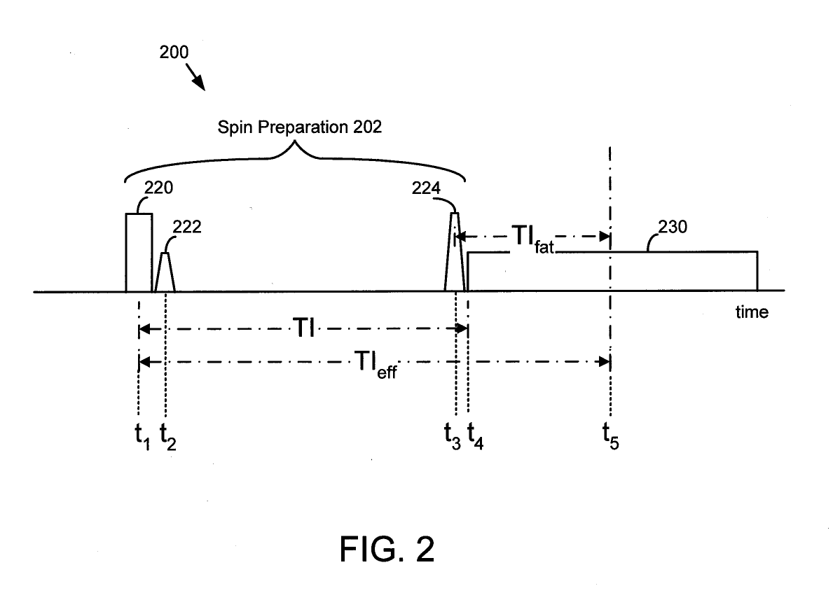

A spin preparation may be used to suppress signal from both fat and a second tissue with the above-described MR system, or any similar or equivalent system for obtaining MR images. FIG. 2 is a schematic diagram of an exemplary pulse sequence that includes a spin preparation comprising a fat-selective saturation RF pulse and a fat-selective inversion RF pulse in accordance with an embodiment. In FIG. 2, the RF aspect of an exemplary pulse sequence 200 is shown. Pulse sequence 200 includes a spin preparation 202 and a base sequence 230. The spin preparation 202 is comprised of inversion and saturation RF pulses which suppress the MR signals from both fat and the second tissue. The base sequence 230 may comprise a single excitation RF pulse and an acquisition window, or may comprise multiple excitation RF pulses and acquisition windows, as for example, in a fast gradient recalled echo (fGRE) acquisition. Spin preparation 202 may be compatible with base pulse sequences such as, for example, a two dimensional (2D) fGRE sequence, a regular 2D or three dimensional (3D) gradient recalled echo (GRE) sequence (in which a single alpha pulse is played out and a single k-space line is acquired following the spin preparation), a fast 3D GRE sequence, a regular spin echo sequence, or a fast spin echo sequence. FIG. 2 shows a spin preparation 202 that is comprised of RF pulses including an inversion RF pulse 220, a fat-selective saturation RF pulse 222, and a fat-selective inversion RF pulse 224, and also shows the timing of these RF pulses relative to the base sequence 230. To create an MR image, the sequence of RF pulses shown in pulse sequence 200, together with appropriate gradient waveforms (not shown), may be played out (i.e., performed or applied) repeatedly until enough data is acquired to reconstruct an image. Multiple frames of data corresponding to individual lines in k-space may be collected during each base sequence 230 by playing out multiple excitation pulses and acquisition windows in the base sequence 230.

The inversion RF pulse 220 is preferably a non-selective 180° inversion pulse that inverts the longitudinal magnetization for all tissues including fat and the second tissue. The inversion RF pulse 220 is played out at time t₁. The starting edge of the base sequence 230 at time t₄ is offset from the inversion RF pulse 220 by a time delay, TI. Time delay TI is set (e.g., prescribed by a user) such that the magnetization from the second tissue achieves its null at time t₅ while the base sequence is being played out. It is preferable that the central lines of k-space are acquired at or near time t₅ while the second tissue is nulled. TIeff is the name given to the time delay between the inversion RF pulse 220 and the acquisition of the central lines of k-space.

The inversion RF pulse 220 is followed at time t₂ by fat-selective saturation RF pulse 222 which may be a fat-selective 90° saturation RF pulse. The longitudinal magnetization of fat (M_{z}^{fat}) is forced to zero by the fat-selective saturation RF pulse 222, while the longitudinal magnetization of the second tissue (M_{z}^{second tissue}) is minimally affected. The fat-selective saturation RF pulse 222 saturates only the fat magnetization, i.e., it drives the spin population of the fat tissue into a state which has an equal number of spins aligned with and against the positive z axis (+z), so that there is no net fat magnetization along the z axis. The purpose of the fat-selective saturation RF pulse 222 is to cause the longitudinal magnetization from fat tissue to be in a known state at a known time point in the pulse sequence 200. The rate of recovery of fat magnetization is known. Thus, the state of the fat magnetization may be determined at any point during the pulse sequence 200 after the reference time point, t₂. The value of M_{z}^{fat} can be determined at the time of a third RF pulse, a fat-selective inversion RF pulse 224, i.e., at time t₃. An inversion flip angle for the fat-selective inversion RF pulse 224 may be chosen such that fat achieves its null at approximately the same time as the second tissue. The inversion flip angle for the fat-selective inversion RF pulse 224 may be determined using equations and methods generally known in the art. Preferably, an acquisition scheme will be used that acquires the central lines of k-space when both the fat tissue and the second tissue are at or near their null points. Examples of acquisition schemes that are compatible with this spin preparation are a “centric encoding scheme”, in which the central lines of k-space are acquired early in the base sequence or a “sequential encoding scheme”, in which the central lines of k-space are acquired near the middle of the base sequence. The null points of fat and the second tissue may be timed to coincide with the acquisition of the central lines of k-space by appropriate modification of the TI, and the flip angle of the fat-selective inversion pulse 224.

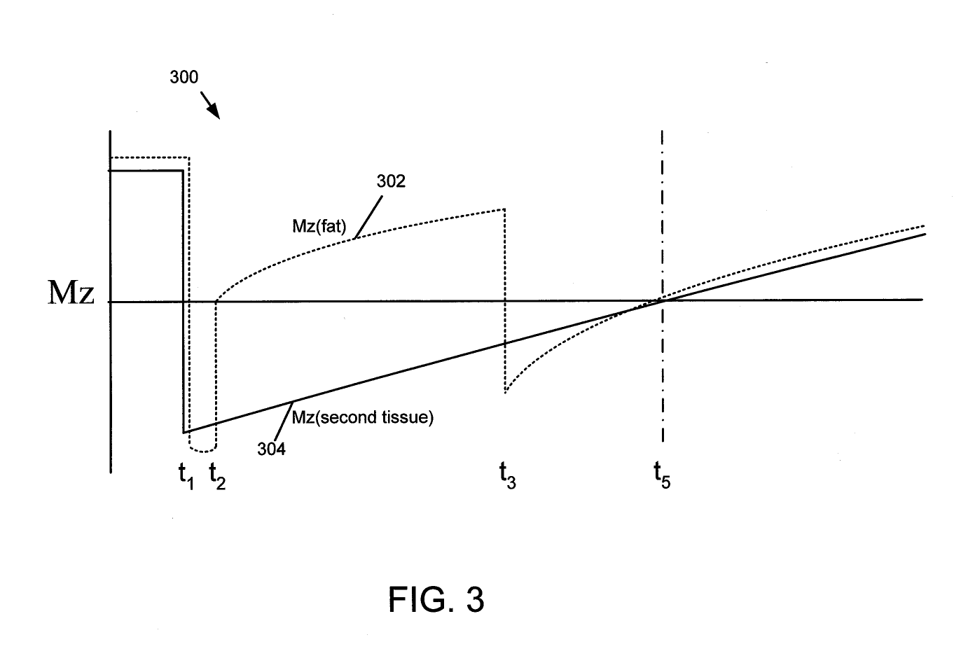

FIG. 3 is a graph of the resulting longitudinal magnetization M_{z} for fat and for a second tissue in accordance with the spin preparation illustrated in FIG. 2 in accordance with an embodiment. The graph 300 in FIG. 3 shows the time evolution of the longitudinal magnetization from fat 302 and from the second tissue 304 in response to the sequence of RF pulses shown in FIG. 2. At time t₁, the inversion RF pulse 220 (shown in FIG. 2) inverts the magnetization from all tissues, including fat and the second tissue. At time t₂, a fat-selective saturation RF pulse 222 (shown in FIG. 2) forces the longitudinal magnetization from fat (M_{z}(fat) 302) to zero, while having minimal effect on the longitudinal magnetization from the second tissue (M_{z}(second tissue) 304). Between time t₂ and t₃, the spins from fat and the second tissue relax to their rest state, and the longitudinal magnetization of fat 302 and the second tissue 304 re-grow along +z. At time t₃, a fat-selective inversion RF pulse 224 (shown in FIG. 2) inverts the fat magnetization. After the fat-selective inversion RF pulse 224, the magnetization from fat re-grows along +z. The flip angle for the fat-selective inversion RF pulse 224 may be chosen such that the null point for fat occurs at time t₅, i.e., at approximately the same time as the null point for the second tissue. The base sequence 230 (shown in FIG. 2) may be determined such that the central lines of k-space are acquired at or near time t₅ when the magnetization from fat is at its null point and the second tissue is at its null point.

Computer-executable instructions for performing a spin preparation according to the above-described method may be stored on a form of computer readable media. Computer readable media includes volatile and nonvolatile, removable and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which can be used to store the desired instructions and which may be accessed by MRI system 10 (shown in FIG. 1), including by internet or other computer network forms of access.

As mentioned above, the spin preparation described with respect to FIGS. 2 and 3 is compatible with various base pulse sequences. In addition, the spin preparation may be applied in various imaging applications such as, for example, cardiac imaging, abdominal imaging, musculoskeletal imaging, or imaging for any other part of the body. Accordingly, the spin preparation 202 (shown in FIG. 2) may be applied to null the signal from fat and a second tissue for any MRI imaging application.

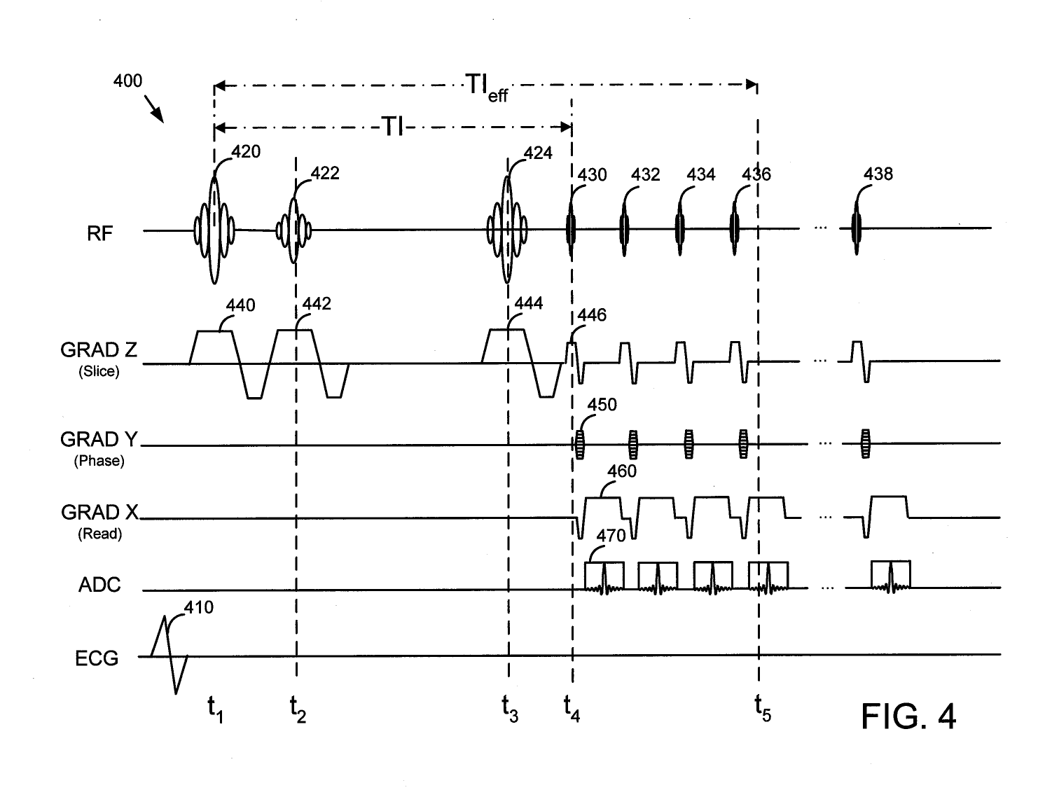

FIG. 4 shows a pulse sequence diagram of an exemplary 2D fast gradient echo pulse sequence that uses the spin preparation illustrated in FIG. 2 to suppress the MR signals from fat and healthy myocardial tissue in accordance with an exemplary embodiment. The pulse sequence 400 is an exemplary ECG-triggered, 2D fast gradient-recalled echo acquisition that may be used to create myocardial delayed enhancement images with suppression of both fat and healthy myocardial tissue. In this embodiment, a 2D fGRE pulse sequence with a segmented k-space acquisition is used as the base sequence. An ECG trigger 410 signals the scanner to start the sequence of pulses shown in pulse sequence 400. FIG. 4 shows RF and gradient pulses corresponding to a single R-R interval, which is the time between successive R waves in an ECG cycle, i.e., the duration of a heartbeat. Because the heart rate is not constant, the variable R-R interval affects magnetization recovery after the acquisition window. For this cardiac application, there is particular need for a saturation pulse to set the longitudinal magnetization from fat to the same starting value on each heartbeat. At time t₁, the first RF pulse played out (i.e., performed or applied) is a slice-selective 180° inversion pulse 420, that inverts the longitudinal magnetization for all tissues in the selected slice. A slice-select gradient and refocusing lobe 440 are also played out on the z-axis at the same time as RF pulse 420. A fat-selective 90° saturation RF pulse 422 is played out at time t₂ along with a slice-select gradient and refocusing lobe 442. After a time delay determined by the choice of TI, a fat-selective inversion RF pulse 424 is played out at time t₃, along with a slice-select gradient and refocusing lobe 444. The flip angle for inversion RF pulse 424 is determined such that the null point for fat coincides approximately with the acquisition of the central lines of k-space. In an alternative embodiment, non-selective RF pulses could replace the slice-selective pulses 420, 422 and 424, requiring no accompanying gradient pulses. At time t₄, an alpha (small flip-angle excitation) pulse 430 is played out with a slice-select gradient and refocusing lobe 446. In this exemplary pulse sequence, a segmented acquisition of k-space data is used, and a train of alpha pulses, 432, 434, 436, 438, is played out as part of the base sequence. Each alpha pulse is followed by an acquisition window (e.g. acquisition window 470), in which the preamplifier 64 (shown in FIG. 1) is connected to the RF coil 56 (shown in FIG. 1). A read gradient and pre-winder (e.g., read gradient and pre-winder 460) is played out during the data acquisition. Immediately preceding each acquisition window, a phase-encoding gradient (e.g. phase-encoding gradient 450), is played out on the y-axis. FIG. 4 shows the null points for fat and for healthy myocardial tissue occurring at the time of the fourth alpha pulse, when the central lines of k-space are acquired. The size of the phase-encoding gradients varies for each acquisition window, and also for each R-R interval, in order to acquire all the necessary data to complete a 2D k-space for the slice. Note that the x, y, and z axes shown in FIG. 4 are logical axes for describing the pulse sequence, and may correspond to any physical direction.

The longitudinal magnetization from infarcted tissue is not affected by the fat-selective RF pulses 422 and 424, and recovers much faster than the healthy myocardium, such that at the time of the first excitation pulse, there is significant longitudinal magnetization from the infarcted tissue available for excitation. Thus, the infarcted tissue will appear enhanced in brightness relative to the healthy myocardial tissue, improving visualization of the infarcted tissue's boundaries.

In various embodiments, k-space may be acquired in a sequential manner, a segmented manner, or any other ordering not described herein may be used. In each case, it is preferable that the flip angle of the fat-selective inversion pulse 424 be determined such that the null point of fat occurs whenever the central lines of k-space are acquired. Without the saturation pulse, the magnetization of fat at the time of inversion pulse 424 would not be known accurately, due to its dependence on the starting magnetization at the ECG trigger 410, which, in turn, depends on the patient's heart rate.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

I claim:

1. A method for generating a magnetic resonance image, the method comprising:
applying a magnetic field to a subject, the subject comprising a plurality of tissues including fat tissue and a second tissue, wherein the magnetic field causes a net longitudinal magnetization in the plurality of tissues including the fat tissue and the second tissue;
generating a first inversion radio frequency pulse configured to invert the longitudinal magnetization from the plurality of tissues including the fat tissue and the second tissue;
generating a saturation radio frequency pulse configured to saturate the longitudinal magnetization of the fat tissue;
generating a second inversion radio frequency pulse configured to invert the longitudinal magnetization of the fat tissue;
generating a first excitation radio frequency pulse; and
acquiring magnetic resonance imaging data.

2. A method according to claim 1, further comprising waiting for a time delay between generating the first inversion pulse and generating the first excitation pulse, wherein the time delay is selected such that the magnetic resonance imaging data is acquired when a longitudinal magnetization of the second tissue is at or near a null point.

3. A method according to claim 2, wherein acquiring the magnetic resonance imaging data is performed when the longitudinal magnetization of the fat tissue is at or near a null point and the longitudinal magnetization of the second tissue is at or near a null point.

4. A method according to claim 1, wherein generating the excitation pulse and acquiring magnetic resonance imaging data are repeated to acquire multiple k-space lines of magnetic resonance imaging data.

5. A method according to claim 4, further comprising waiting for a time delay between generating the first inversion pulse and generating the first excitation pulse, wherein the time delay is selected such that k-space lines of magnetic resonance imaging data corresponding to a central aspect of k-space are acquired when a longitudinal magnetization of the second tissue is at or near a null point.

6. A method according to claim 5, wherein the second inversion radio frequency pulse inverts the longitudinal magnetization from the fat tissue by a pre-determined angle, such that the k-space lines of magnetic resonance imaging data corresponding to the central aspect of k-space are acquired when the longitudinal magnetization of the fat tissue is at or near a null point and the longitudinal magnetization of the second tissue is at or near a null point.

7. A method according claim 1, wherein the second tissue is healthy myocardial tissue.

8. A method according to claim 4, wherein generating the excitation pulse and acquiring magnetic resonance imaging data are performed in accordance with a fast gradient recalled acquisition.

9. A computer-readable medium having computer-executable instructions for performing a method for generating a magnetic resonance image, the computer-readable medium comprising:
program code for generating a first inversion radio frequency pulse configured to invert a longitudinal magnetization from a plurality of tissues in a subject including a fat tissue and a second tissue;
program code for generating a saturation radio frequency pulse configured to saturate the longitudinal magnetization of the fat tissue;
program code for generating a second inversion radio frequency pulse configured to invert the longitudinal magnetization of the fat tissue;
program code for generating a first excitation radio frequency pulse; and
program code for acquiring magnetic resonance imaging data.

10. A computer-readable medium according to claim 9, wherein acquiring the magnetic resonance imaging data is performed when the longitudinal magnetization of fat is at or near a null point and the longitudinal magnetization of the second tissue is at or near a null point.

11. A computer-readable medium according to claim 9, wherein generating the excitation pulse and acquiring magnetic resonance imaging data are repeated, such that multiple k-space lines of magnetic resonance imaging data are acquired.

12. An apparatus for generating a magnetic resonance image, the apparatus comprising:
a magnetic resonance imaging assembly comprising a magnet, a plurality of gradient coils, a radio frequency coil, a radio frequency transceiver system, and a pulse generator module; and
a computer system coupled to the magnetic resonance imaging assembly and programmed to perform a pulse sequence comprised of:
a first inversion radio frequency pulse configured to invert a longitudinal magnetization from a plurality of tissues in a subject including a fat tissue and a second tissue;
a saturation radio frequency pulse configured to saturate a longitudinal magnetization of the fat tissue;
a second inversion radio frequency pulse configured to invert the longitudinal magnetization of the fat tissue;
a first excitation radio frequency pulse; and
an acquisition window to acquire magnetic resonance imaging data.

13. An apparatus according to claim 12, wherein the pulse sequence further comprises a time delay after the first inversion pulse, the time delay selected such that the acquisition window is performed at or near a null point of a longitudinal magnetization of the second tissue.

14. An apparatus according to claim 13, wherein the second inversion radio frequency pulse is set to apply a pre-determined flip angle to the longitudinal magnetization of the fat tissue such that the acquisition window is performed at or near a null point of the longitudinal magnetization of the fat tissue and at or near a null point of the longitudinal magnetization of the second tissue.

15. An apparatus according to claim 12, wherein the pulse sequence further comprises a plurality of excitation pulses and acquisition windows configured to acquire multiple k-space lines of magnetic resonance imaging data.

16. An apparatus according to claim 15, wherein the pulse sequence further comprises a time delay after the first inversion pulse such that at least one acquisition window corresponding to a central k-space line is performed at or near a null point of the longitudinal magnetization of the second tissue.

17. An apparatus according to claim 16, wherein the second inversion radio frequency pulse is set to invert the longitudinal magnetization from the fat tissue by a pre-determined angle, such that at least one acquisition window corresponding to a central k-space line is performed at or near a null point of the longitudinal magnetization of the fat tissue and at or near a null point of the longitudinal magnetization of the second tissue.

18. An apparatus according to claim 17, wherein the pulse sequence includes a fast gradient recalled echo acquisition.
